(19) United States
(12) Patent Application Publication
(10) Pub. No.: US 2015/0118707 A1
(43) Pub. Date: Apr. 30, 2015

(54) METHOD AND DEVICE FOR DETECTING METABOLLICALLY ACTIVE CELLS

(71) Applicant: Ponnambalam Selvaganapathy, Dundas (CA)

(72) Inventor: Ponnambalam Selvaganapathy, Dundas (CA)

(21) Appl. No.: 14/524,454

(22) Filed: Oct. 27, 2014

Related U.S. Application Data
(60) Provisional application No. 61/895,531, filed on Oct. 25, 2013.

(51) Int. Cl. C12Q 1/04

(52) U.S. Cl. CPC C12Q 1/04
USPC 435/34

(57) ABSTRACT

A method for the detection of metabolically active cells using microwells is provided. A sample containing one or more cells is segmented into a plurality of microwells each containing a metabolic indicator. The microwells are then monitored for a change in the level or activity of a metabolic indicator, thereby indicating the presence or absence of metabolically active cells in each microwell. Also provided are methods for the detection of cells such as bacteria that are resistant to antibiotics or for the detection of particular cell types such as mycobacteria. Also provided are screening methods for identifying compounds with an effect on cell growth or metabolism.

### RELATED APPLICATIONS

This application claims priority to U.S. Provisional Patent Application Ser. No. 61/895,531 filed Oct. 25, 2013, the entire contents of which are hereby incorporated by reference.

### FIELD OF THE INVENTION

The following invention relates to a method and device for detecting cells and more specifically to a method and device for detecting metabolically active cells using a multi-well plate.

### BACKGROUND OF THE INVENTION

Infectious diseases are a leading cause of death in the world even today. According to the WHO, in 2008, infectious diseases such as respiratory infections, gastroenteritis, HIV/AIDS and tuberculosis (TB) alone were responsible for ˜12 million deaths. Although these infectious agents are well known and suitable drug exist to treat them, the recent emergence of drug resistant pathogens is of grave concern. These factors are likely to make infectious diseases, its diagnosis and treatment as a top health priority in both the developing and the developed worlds in the coming decades.

Accumulating evidence of research suggests that early detection and treatment of bacterial contamination is the most efficient prevention strategy to an outbreak. The fast and accurate detection of bacteria or other pathogens is vital for public health, food and water safety, medical diagnosis of diseases and in biological warfare defense.

### Tuberculosis

Nearly one-third of the world's population is infected with the TB bacillus (Mycobacterium tuberculosis) [1]. In 2009, 1.7 million people died from the disease globally and 9 million more are infected each year [2]. Asia and Africa are disproportionately affected. India, China, South Africa, Nigeria, and Indonesia lead the list of number of infected cases [2]. According to World Health Organization (WHO), around 2 million people in India develop TB and 300,000 die of it each year.

Although drugs have been developed to combat the disease, accurate, fast and definite diagnosis at an early stage is a problem. Furthermore, multidrug resistant (MDR) and extensively drug resistant (XDR) mycobacterium have emerged recently, complicating the treatment. In MDR the TB bacteria is resistant to at least isoniazid and rifampicin. In case of XDR tuberculosis it is also resistant to fluoroquinolone and one of three injectable aminoglycosides (capreomycin, kanamycin, and amikacin) [2].

A ministerial meeting on high M/XDR-TB burden countries, organized by the WHO estimates that 60 million culture investigations and five million drug susceptibility testing (DST) investigations are required annually to meet the diagnostic goals of drug-resistant and HIV-associated TB by 2015, resulting in a current gap of 50 million cultures and 4.5 million DSTs per annum, 85% of these in the high burden MDR-TB countries [3].

The current diagnostic process is multi-layered, accumulative, resource intensive and takes long time. Bacterial culture is the gold standard but takes 4-8 weeks. Alternate methods such as staining, immunoassays and nucleic acid assays are faster but are either not definitive or expensive and don't provide all the information sought by the medical professional in formulating a treatment and saving lives.

### Staining

Diagnosis of TB started in 1882 when Robert Koch discovered a method for staining M Tuberculosis bacillus [4]. He devised a method for culturing the bacteria using a solid medium of cattle blood serum. Then, he exposed it to alkaline methylene blue for 24 hours which revealed fine rod like shape of the bacteria. Hearing Koch's lecture, Paul Ehrlich obtained some pure bacterial cultures and experimented with various modifications. He stained only for 15-30 min and then used 30% nitric acid with alcohol to remove the stain from the surrounding tissue. M Tuberculosis bacillus retains the stain and was not discoloured. He also counterstained the remaining tissue and heated the slide to improve the contrast [5]. This method with modifications from Ziehl and Neelsen became what is known as the Acid Fast Bacillus (AFB) staining method [6][7].

Nowadays, sputum samples are collected, smeared onto a glass slide, dried and heated to fix it, rinsed in carbol fuchsin, dried, washed with hydrochloric acid and then counter stained with methylene blue. The entire process can be done in a few hours. The cell wall of mycobacteria and other acid fast bacteria is composed of long chain carboxylic acids called mycolic acids. The cell wall possesses a significant barrier to both the entry as well as the elution of any dye that is staining the sample. The use of a lipophillic agent and heating enabled the initial penetration of the dye into the cell. Upon washing with acid-alcohol solutions in the absence of a lipophillic agent and heat, the dye from other tissues are easily eluted while those in the acid fast bacteria are not as the cell wall is reconstituted.

Staining is inexpensive and fast but non-specific as other bacteria that have similar cell wall properties can be present. A positive AFB is only taken as a indicator for more comprehensive testing.

### Immunoassay

A positive AFB combined with a suspicious chest X-ray then motivates the doctor to order a Mantoux tuberculin test which probes for the body's reaction to antigens of the tubercle bacillus. However, this test is not sensitive, results in a lot of false positives and takes several days to obtain results. Also, persons who previously had a BCG immunization are likely to indicate positive. Alternatively, a Interferon-γ release assay test can be performed which measures the immune reaction in response to M. Tuberculosis specific antigens. This is a more specific test and not affected by BCG vaccination. However, it cannot distinguish between latent and active TB. Considering that a wide population has latent infection but in not at risk for transmission, the test is not considered as the gold standard.

### Nucleic Acid Based Detection

The nucleic acid of an organism contains certain sequences that are unique to that organism. Identification of the presence of these sequences can be used for detection of that organism. These unique sequences were identified from either the ribosomal RNA of the organism which are conserved or from mycobacteria specific antigens that were known. The main advantages of the nucleic acid based testing methods were 1) They accelerated the process of identification of pathogens and, 2) They made possible identification of species that were not culturable.

Initially, in the late 1980s, hybridization probes were designed [8] that were either radio/enzyme labeled or had an avidin attachment take could be labeled later. These probes would hybridize the denatured DNA present in the sample and when isolated, indicate the presence of the specific nucleic acid and by extension the organism of interest. However, they suffer from poor sensitivity as 10⁶ organisms were required for identification. An amplification step by culture before using the molecular method improved sensitivity but also increased the time for analysis from a few hours to days.

Very soon, in 1990, the amplification step was also performed using a molecular biology method of Polymerase chain reaction (PCR). PCR was developed in 1983 to amplify a specific sequence of DNA that may be present in the sample by copying it in an exponential process. Very soon after its development, PCR was applied for amplification and detection of specific sequences that were unique to M. Tuberculosis bacillus (65 kDa heat shock gene). A 65 kDa heat shock gene was used as the unique marker for PCR amplification and mycobacteria identified with limits of detection of 100 bacterial cells [9]. Soon, other unique sequences specific to M. Tuberculosis were identified and used on PCR to detect as low as 20 cells [10]. Almost immediately, detection in clinical samples using PCR method was also demonstrated [11], [12].

The greatest advantage of PCR—its ability to detect very small numbers of bacteria—also proved to be its drawback. Contamination during sample preparation and through reusable equipment led to a large number of false positives [13]. Since then, over the past 15 years, standardization of the protocol and automation of sample preparation and handling has led to a highly specific and sensitive test with very low false positives. Automated PCR based test for Tuberculosis has been recently commercialized by Cepheid Corp. Its Gene Xpert System is an automated sputum processing and real-time PCR device that can process and detect MTB in 2 hrs. It has a limit of detection of 131 CFU/ml of sputum with very high specificity [14]. A large multi-national trial has also been conducted on this system which determined sensitivities of 72·5% when processing one sputum specimen, 85·1% when processing two, and 90·2% when processing three for sputum smear-negative disease [15]. Specificity of 99·2% was also obtained.

Although, this technique shows excellent promise for clinical settings it is still very expensive. According to Foundation for Innovative New Diagnostics (FIND), the price for these systems currently are US$55,000-62,000 and cost of each cartridge is US$55-120. FIND has negotiated discounts of 75% to provide the instrument at US$17,000 and the cartridge at US$17. These still represent substantial costs. The reason for the high cost is due to expensive reagents (which include primers, polymerase enzyme as well as other chemicals) that are needed, complex instrumentation needed to perform unit operations such as lysis, DNA extraction and amplification as well as the need to prevent non-specific contamination (This is a big problem in PCR-especially when the lab is processing positive samples which result in amplification of the TB specific DNA [13]). In addition, it does not provide information on the viability of the bacteria and its drug resistance to multiple drugs that are available to treat it.

Therefore, in the end, multiple tests get ordered serially over extended period of time in the face of inconclusive evidence. The diagnosis is accumulative and non-definitive. Simultaneously, the doctor orders a bacterial culture test as it is the gold standard to determine the presence, viability and drug resistance of the Tuberculosis bacillus. The culture test is trusted by the professionals in the field as it gives the physician exactly the information that they seek in formulating a treatment plan, namely: Is it TB? Is it viable? Is it drug resistant?

### Alternate Approaches

At its fundamental level bacterial culture is a simple yet robust method to identify that a particular organism is alive (viable) and to visualize it to the naked eye through amplification (colony growth). Our visual resolution then determines the smallest colony that we can see and hence the number of divisions that the bacteria has to go through. In the case of the slow dividing mycobacteria, which divides every 15-18 hours, this process is long and hence the assay takes several weeks.

However, there are other methods that one could use to determine viability and growth. Any organism that is alive will consume nutrients and excrete waste. Thus by measuring the material that is consumed or excreted, one could have an early indicator of the viability of the bacteria even before it has divided and grown sufficiently to be visually noticed. This is the principle behind the Mycobacteria Growth Indicator Tube (MG IT).

In this method, a growth medium that is specific for mycobacteria (Middlebrook 7H9 broth base) along with an enrichment media (containing Oleic acid, Albumin, Dextrose and Catalase) and antimicrobial media (containing Polymyxin B, Amphotericin B, Nalidixic Acid, Trimethoprim, Azlocillin) is added to the digested sputum sample placed in a silicone coated transparent tube. This liquid medium provides a specific condition for the growth of mycobacteria while preventing other contaminating species from growing. The silicone coating in the tube is impregnated with a fluorophore (tris 4,7-diphenyl-1,10-phenonthroline ruthenium chloride pentahydrate) that does not fluoresce in the presence of oxygen. Since mycobacteria is aerobic, it consumes oxygen that is present in the medium during metabolism and depletes the oxygen in the surrounding environment, producing fluorescence in the silicon layer. The presence of 10⁵⁻⁶ CFU/ml is the cutoff for observation of the fluorescence in the conventional MGIT tube which contains 7 ml of broth. Positive growth in 8-14 days are common in liquid medium such as the ones used in the MGIT. Drug resistance can be probed by adding the appropriate drug to the broth and measuring growth through fluorescence.

This is a simple, elegant and robust method for determining whether a specific bacteria (M. Tuberculosis) is 1) present, 2) viable and 3) drug resistant. It transduces this biochemical information of the metabolism of the bacteria into an optical signal that could be read out using a simple UV lamp and a fluorimeter. This method does not require expensive reagents, nor lysis and processing of the cells as in the case of PCR. For sputum samples it needs digestion similar to PCR. Although this method is considerable shorter than the solid media cell culture it is still long for rapid diagnostic purposes. An ideal diagnostic system will provide information on the identity of the bacteria, its viability, and drug resistance, while simultaneously having fast detection times and require minimal sample preparation.

### SUMMARY OF THE INVENTION

In one aspect, there is provided a method and device for the detection of metabolically active cells such as bacteria. One advantage of the present invention is the detection of cells that are metabolically active and not dead cells that may otherwise appear similar to live cells.

In one embodiment, the inventor has developed a miniaturized and parallelized method for the rapid detection of cells in a biological sample by detecting and/or measuring the metabolism of the cells such as by detecting the consumption of nutrients or oxygen or the production of waste products by the cells. In one embodiment, a sample is separated into many small volumes prior to detecting a change in the level or activity of a metabolic indicator within the small volume such as the consumption of a nutrient or the production of a waste product. As set out in the Examples, the inventor has been successful in detecting E. Coli bacteria as a model pathogen. In one embodiment, there is also provided a simple mechanical processes to take a larger sample (e.g. 0.5-1 ml) and segment it into a numer of smaller samples. This process is simple, does not require any electrical power and can be quickly completed. It produces precise segmentation into appropriate volumes that have been designed in the structure and can be used with a wide variety of sample types containing a variety of biological materials such as cells, DNA, proteins, lipids and other particulate matter.

Optionally, the methods and devices described herein use a metabolic indicator within each microwell to sense the metabolic activity of one or more cells within the microwells. In one exemplary embodiment, the inventor has demonstrated monitoring and/or detecting oxygen consumption in these small volumes by measuring the fluorescence of an oxygen sensitive fluorophore in solution. As set out in the Examples, the effective concentration of the cells in some of the small subdivided volumes is significantly higher than the original sample solution, while in the other volumes it is much smaller and close to zero. Therefore, in those volumes that have one or more cells present, the cells are able to consume the limited supply of the nutrients and/or oxygen much faster than in the large sample. Since the consumption of nutrients and/or oxygen by cells in a subdivided volume occurs much faster than in large sample, in some embodiment the method detects cells such as bacterial pathogens orders of magnitude faster than in a bulk undivided sample.

In one aspect, the method and device described herein may also be used to determine the susceptibility or resistance of cells such as bacteria to antibiotics and/or other test agents. Optionally, the methods described herein further include selecting a therapy for a subject based on detecting the presence or absence of cells that are resistant or susceptible to a particular antibiotic or therapy. Similarly, the methods and devices described herein may be used in screening assays to determine whether cells or bacteria in a sample are resistant or susceptible to a particular antibiotic or test agent or to identify test agents with antibiotic activity. As set out in the Examples, including an antibiotic in the microwells along with the test cells produced a lower rate of consumption of oxygen which may be used as a method to measure the efficacy of drug or test compound against cells in the sample.

Furthermore, as set out in the Examples, the inventor has demonstrated that the method and device described herein may be used to estimate the concentration or number of cells in a sample. In one embodiment, the magnitude of the change in the metabolic indicator is indicative of the concentration of cells in the sample. In one embodiment, the relative number of microwells with a detectable change in the metabolic indicator is indicative of the concentration of cells in the sample.

Accordingly, in one aspect there is provided a method for detecting the presence of metabolically active cells in a sample. In one embodiment the method comprises separating the sample into one or more microwells and detecting a change in a level or activity of a metabolic indicator in one or more of the microwells, where the change is indicative of the presence of one or more metabolically active cells in the sample.

In one embodiment, the metabolic indicator is a compound that is produced by the metabolically active cells and an increase in the level of the metabolic indicator is indicative of the presence of one or more metabolically active cells in the sample. In one embodiment, the metabolic indicator is a compound that is consumed by the metabolically active cells, such as oxygen (for aerobic cells) or a nutrient in a growth medium, and a decrease in the level of the metabolic indicator is indicative of the presence of one or more metabolically active cells in the sample.

In some embodiments, the metabolic indicator is an exogenous compound and the methods described herein include detecting the presence of the exogenous compound or part of the exogenous compound that reacts with the metabolically active cells within the microwells. For example, in one embodiment, the metabolic indicator is an oxygen-sensitive fluorophore and detecting a change in the level of the oxygen-sensitive fluorophore is used to detect a change in the level of oxygen in the one or more microwells.

In some embodiments, the metabolic indicator is a substrate for an enzyme produced by the metabolically active cells and a change in the level of the substrate is indicative of the presence of metabolically active cells in the sample.

The method used to detect the level or activity of the metabolic indicator will depend on the particular metabolic indicator. For example, in some embodiments the metabolic indicator contains a fluorophore or a fluorescent label and the level of the metabolic indicator is determined using optical methods. In some embodiment, the level or activity or the metabolic indicator is converted into an electrical, optical, chemical or thermal signal inside the microwells. In some embodiments, the metabolic indicator is detectable by spectroscopy, electrochemical analysis, immunohistochemistry, chromatography or mass spectrometry.

In some embodiments, the level of the metabolic indicator is zero or presumed to be at a certain level within the microwell prior to separating the sample into the microwells. Detecting the presence of the metabolic indicator in one or more microwells after the sample has been separated into the microwells is therefore indicative of a change in the level of the metabolic indicator and the presence of metabolically active cells within the sample.

In some embodiments, the methods described herein include detecting a change in the level or activity or two or more, three or more or 4 or more metabolic indicators.

In some embodiments, the methods described herein involve monitoring the level of a metabolic indicator over time in order to detect a change in the level of the metabolic indicator. For example, in one embodiment, the methods comprises:
determining a first level or activity of the metabolic indicator at a first time point;
determining a second level or activity of the metabolic indicator at a second time point; and
comparing the first level or activity at the first time point with the second level or activity at the second time point, wherein an increase or decrease in the level or activity of the metabolic indicator indicates the presence of one or more metabolically active cells in the sample.

Optionally, the methods described herein comprise determining a first level or activity of the metabolic indicator at a first time point for a plurality of microwells; determining a second level or activity of the metabolic indicator at a second time point for a plurality of microwells; and comparing the first level or activity at the first time point for the plurality of microwells with the second level or activity at the second time point for the plurality of microwells. In some embodiments, the methods described herein include determining a change in the level of activity of a metabolic indicator in at least 5 microwells, at least 10 microwells, at least 50 microwells, at least 100 microwells, at least 250 microwells or at least 500 microwells.

In one embodiment, the second time point is less than 48 hours, less than 30 hours, less than 24 hours, less than 12 hours, or less than 6 hours from the first time point. In one embodiment, the second time point is less than 4 hours, less than 2 hours, less than 1 hour, less than 30 minutes or less than 15 minutes from the first time point.

In one embodiment, a change in the level or activity of a metabolic indicator in at least one microwell is indicative of the presence of metabolically active cells in the sample. Optionally, a change in the level or activity of a metabolic indicator in a plurality of microwells is also indicative of the presence of metabolically active cells in the sample. In some embodiment, a change in the average level of the metabolic indicator in a plurality of microwells over time is indicates the presence of one or more metabolically active cells in the sample. In some embodiments, the magnitude of the change in the level or activity of the metabolic indicator is indicative of the level or concentration of cells in the sample.

In one embodiment, a sample comprising one or more cells or suspected of comprising one or more cells is separated into one or more microwells. In one embodiment, the microwells are large enough to contain one or more cells and small enough such that the metabolic activity of the cells within the microwell is sufficient to detectably change the composition of any media or fluid within the microwell. In one embodiment, each microwell has a volume between 1 picoliter and 1 microliter. In one embodiment, each microwell has a volume between 0.1 nanoliters and 1000 nanoliters. In one embodiment, each microwell has a volume between 1.0 nanoliters and 100 nanoliters or between 1.0 nanoliters and 1 microliter.

In one embodiment, the sample is separated into the one or more microwells by contacting the top surface of the plate with the sample and moving all or part of the sample across the top surface of the plate over the one or more microwells. Other methods for separating a fluid sample into microwells known in the art of microfluidic technologies, such as ink jet printing etc. may also be used to separate the sample.

In one embodiment, the one or more microwells are on a microfluidic device. Optionally, the microfluidic device comprises a plate with a top surface and the one or more microwells extend below the top surface. In one embodiment, the top surface is hydrophobic. In one embodiment, the microwells extending below the top surface are hydrophilic. In one embodiment, the microfluidic device is made of a material that is amenable to surface modification, such as to modify the surface of the microwells with a material that is hydrophilic or the top surface of the microfluidic device with a material that is hydrophobic.

In one embodiment, the methods and devices described herein include a microfluidic device comprising a plurality of microwells in an array. In one embodiment, the array comprises between 1 and 10⁸ microwells. In one embodiment, the array comprises between 5 and 10,000 microwells, optionally between 10 and 5,000 microwells. In one embodiment the array comprises at least 5, 10, 20, 50, 100, 200, 500 or 1000 microwells. In one embodiment, the array comprises between 10 and 10⁸ microwells per cm², optionally at least 5, 10, 20, 50, 100, 200, 500, 1000 or 10,000 microwells per cm².

In one embodiment, the microwells contain a growth media or fluid surrounding or in contact with the one or more cells. In one embodiment, the microwells contain a growth media before or after separating the sample into one or more microwells. In one embodiment, the growth media is mixed with the sample prior to separating the sample into one or more microwells.

Optionally, the growth media may be selective for a particular cell type. For example, in one embodiment the growth media is selective for mycobacteria and the presence of one or more metabolically active cells in the sample indicates that the sample contains mycobacteria.

In one embodiment, the growth media comprises one or more antibiotics or test compounds. Optionally, the sample may be known to contain a cell or bacteria (such as a standardized bacterial culture) along with one or more antibiotics or test compounds and the detection of metabolically active cells or the absence of metabolically active cells is indicative of the effect of the antibiotic or test compound on cellular metabolism. In one embodiment, the growth media comprises an antibiotic and the presence of one or more metabolically active cells in the sample indicates that the sample contains cells that are resistant to the antibiotic.

In one embodiment, there is also provided a microfluidic device comprising one or more microwells for the detection of a metabolically active cell as described herein.

Further aspects and advantages of the embodiments described herein will appear from the following description taken together with the accompanying drawings.

### BRIEF DESCRIPTION OF THE DRAWINGS

FIG. 1 is a representation of the miniaturization and compartmentalization of a milliliter volume into several microliter volumes. The concentration of bacteria (red triangles) is small in the large volume. However, the concentration is high is some of the microvolumes while is zero in others.

FIG. 2 shows the device fabrication process and procedure.

FIG. 3 shows selective surface modification of the nano multi-well plates.

FIG. 4 shows the comparison of oxygen diffusion in PDMS (left) and PU (right) from t=0 to t=360 sec.

FIG. 5 shows nano multi-well plates containing microwells with various size from 1 nL to 100 nL filled with DI water (dyed with methylene blue).

FIG. 6 shows nano multi-well plates containing microwells with various size from 1 nL to 100 nL filled with protein-rich solution (milk dyed with methylene blue)

FIG. 7 shows bacteria growth (GFP) inside a 1.5 nL micro well filled with a 0.3 mg/mL RDTP in LB. (a) t=0; (b) t=2.5 hr; (c) t=5 hr

FIG. 8 shows 1 day incubation of GFP bacteria. Initially <10 bacteria were present in the wells.

FIG. 9A shows a modified microtiter plate for placing nano multi-well plate; FIG. 9B shows photography of nano multi-well plate placed inside the modified microtiter plate; FIG. 9C shows original locations of 96 well plate are highlighted in blue circles.

FIG. 10 shows the dynamic fluorescent response for sample solutions with various bacteria concentration using the traditional microtiter plates (sample size: 0.1 mL).

FIG. 11 shows the dynamic fluorescent response from the same sample solution in wells with various sizes.

FIG. 12 shows fluorescent images of single wells by overlapping the fluorescence from RDTP (highlighted in red-not shown), the fluorescence of GFP E. coli (highlighted in green-not shown) and the bright filed image (highlighted in grey-not shown). Sample solutions with a bacteria concentration of (a) 10⁶ per mL and (b) 10⁷ per mL.

FIG. 13 shows the color variation of MB in 1 mL vials after a 24 hr incubation.

FIG. 14 shows the color of vials filled with a range of bacteria concentration from 10³ to 10⁷ per mL after a 24 hr incubation.

FIG. 15 shows discoloration of MB on nano multi-well plate with a well size of 100 nL.

FIG. 16 shows the captured images of FIG. 15 processed using ImageJ to obtain the mean grey scale.

FIG. 17A shows filing an array of 250 μm cubes (6.25 nL); FIG. 17B shows filling an array of 1000 μm cubes (98.0 nL).

FIG. 18 shows the percentage of microwells filled per swipe (with a coverslip) using a mixed solution of de-ionized water and methylene blue for various volumes of micro wells.

FIG. 19 shows the fluorescent intensity of RTDP after 2.5 hours illustrating the oxygen quenching as the oxygen levels decrease within the wells demonstrating the existence of bacterial metabolism.

FIG. 20 shows the results from using confocal microscopy to image the fluorescence of the metabolic indicator RTDP as it is quenched and the increase in relative intensity resulting from the decrease of oxygen per well containing 1-10 bacteria. The wells containing only LB media is plotted against this to compare the unquenching of the fluorophore.

FIG. 21 shows that the volume of the microwells has a significant effect on the detection of metabolic activity. The smaller the well volume (500 nL, 20 nL), the more prominent the changes in oxygen levels within the well. The larger the well, the longer the time it takes to detect changes in oxygen levels (0.1 mL).

FIG. 22 shows the results from using confocal microscopy to image the fluorescent intensity of the RTDP when a drug (ampicilin) is added to the nanowell. When the drug is present in the nanowell, it inhibits bacterial growth, whereas when no drug is present in the well, the E. coli OP50 strain grows naturally and an increase in fluorescence is observed due to oxygen quenching of the metabolic indicator.

### DETAILED DESCRIPTION

In one aspect, there is provided a simple, rapid and sensitive method for the detection of cells in a sample by separating the sample into one or more separate volumes. In one embodiment, the sample is separated into one or more microwells, optionally a plurality of microwells in an array. The methods and devices described herein detect a change in a level or activity of a metabolic indicator in one or more of the separate volumes. In one embodiment, the change is indicative of the presence of one or more metabolically active cells in the sample. By segmenting the sample into separate volumes, the time required for detection of the cells is significantly reduced.

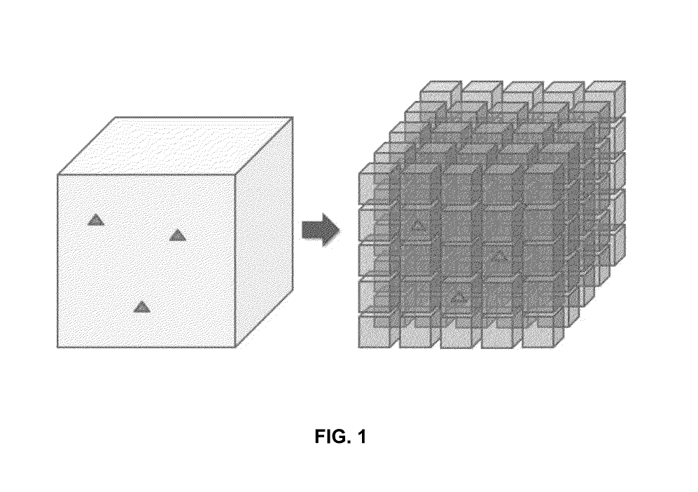

Consider the volume of sample as depicted in FIG. 1. Assume that it is 1 ml, holds oxygen of ˜10 mg/I and contains within it 100 bacteria. Also assume that each bacteria consumes oxygen at the rate of 1×10⁻⁸ mg O₂/hr. A simple calculation shows that it will take 1000 hrs (42 days) for the bacteria to consume all the oxygen. This will lead to fluorescence of the fluorophore and the end point of the detection. Now consider the case when the 1 ml volume is split into million individual 1 nl volumes. Some of these volumes have one bacterium in them and the others don't have any. Each nanoliter volume now contains 10×10⁻⁹ mg of O₂. In the volumes that have the bacteria, calculation shows that the oxygen content is completely consumed in 0.1 hr (6 min), while in others the concentration remains the same. This example demonstrates the power of miniaturization and parallelization. If it is possible to segment a volume of fluid into micro or nanovolumes and analyze for presence of absence of metabolites in parallel across those millions of micro/nanovolumes then the process of detection for viability of bacteria can be accelerated from 1000 hrs to under 6 mins. This strategy can be used for the detection of metabolically active cells such as, but not limited to, M. Tuberculosis bacteria or other pathogens.

As set out in the Examples, the methods and devices described herein have been demonstrated using a laboratory strain of E. coli as a model cell and bacterial pathogen. In one aspect, the method of compartmentalization and parallelization depends on the concentration of the metabolic indicator in individual containers being distinct from each other to recognize the metabolism of the cells. For example, when oxygen is used as a metabolic indicator rapid oxygen diffusion from one chamber to the next dilutes the contrast provided. Thus materials for the device may be selected or have a surface coating that will provide permeability between microwells. In one embodiment, if the metabolic indicator is oxygen or an oxygen sensitive compound the materials of the device should be selected or have a surface coating that provides low oxygen permeability.

As used herein, the term “metabolic indicator” refers to a substance which is produced or consumed by a living cell, or a substance that is directly or indirectly modified by the metabolic activity of a living cell. In one embodiment, the metabolic indicator is a compound that is produced by the metabolically active cells or a compound that is consumed by the metabolically active cells. In some embodiments, the metabolic indicator is an exogenous compound that is added to the sample or is present in the microwells. For example, in one embodiment the metabolic indicator is combined or mixed with the sample prior to separating the sample into the plurality of microwells.

In some embodiments, the level or activity of the metabolic indicator is converted into an electrical, optical, chemical or thermal signal inside the microwells. Optionally, each microwell may have a sensor suitable for the detection of a signal generated within the microwell by the metabolic indicator. In other embodiments, the activity or level of the metabolic indicator is detected using optical techniques such as a spectrometer. For example, in some embodiments the metabolic indicator is a fluorophore and changes in the level of the metabolic indicator are detected by optical methods that detect a change in the emission spectra metabolic indicator within each microwell.

In one embodiment, the metabolic indicator is detectably labeled, such that changes in the level of the metabolic indicator are detected by changes in the level of the detectable label. In some embodiments, the metabolic indicator is altered or modified by the metabolic activity of a cell in order to produce a detectable signal. For example, in one embodiment the cells are aerobic cells that consume oxygen and the metabolic indicator is oxygen or an oxygen sensitive compound.

In one embodiment, the metabolic indicator is an oxygen sensitive fluorophore, wherein the emission spectra of the fluorophore changes in response to oxygen. In one embodiment, the oxygen sensitive fluorophore is selected from the group consisting of a ruthenium-based molecule, a metalloporphyrin-type molecule and a metal halide. Examples of ruthenium-based molecules include ruthenium tris(2,2′-diprydl)dichloride hexahydrate (RTDP), 4,7-diphenyl-1,10-phenathroline ruthenium (II) chloride, ruthenium(II)-tris(1,10-phenanthroline) and dichlorotris(1,10-phenanthroline) ruthenium (II) hydrate. Examples of metalloporphyrin-type molecules include Platinum(II)- and palladium(II)-complexes of octaethyl-porphyrin; palladium (Pd)-coproporphyrin and palladium meso-tetra-(4-carboxyphenyl) porphine. In some embodiments, the metabolic indicator is a metal halide, such as a molybdenum cluster e.g. Mo₆Cl.

In some embodiments, the metabolic indicator is a substrate for an enzyme produced by the metabolically active cells and a change in the level of the substrate is indicative of the presence of metabolically active cells in the sample. In one embodiment the substrate is an exogenous compound that is a substrate specific for enzymes produced by a certain type of bacteria, such that detecting a change in the metabolic indicator is indicative of the certain type of bacteria. For example, in one embodiment, the substrate is 4-metylumbelliferyl-β-D glucuronide (MUG) and an increase in the level of methylumbelliferone is indicative of the presence or one or more metabolically active cells that produce β-glucuronidase, such as coliform bacteria.

Other exemplary metabolic indicators useful with the methods and devices described herein include shiga toxin, Nicotinamide adenine dinucleotide phosphate (NADPH), Nicotinamide adenine dinucleotide (NADH), flavin adenine dinucleotide (FADH), Flavin mononucleotide (FMNH), methylene blue and trypan blue.

As used herein, the term “metabolically active cell” refers to a living cell capable of performing enzyme-catalyzed reactions in order to grow and/or reproduce. In one embodiment, the cell is a bacteria, optionally an anaerobic or aerobic bacteria. In one embodiment, the cell is derived or isolated from an organism or cell culture. In one embodiment, the cell is a fungal cell. In some embodiments, the cell is a pathogen or bacteria associated with infectious disease. In one embodiment, the bacteria is a bacteria associated with foodborne illness. Examples of bacteria include, but are not limited to, Mycobacterium including Mycobacterium tuberculosis, species of Streptococcus and Pseudomonas, Acinetobacter, Bacillus such as Bacillus anthracis, Neisseria such as Neisseria gonorrhoeae or Neisseria meningitidis as well as species of Shigella, Campylobacter, or Salmonella.

In one embodiment, the methods and devices described herein comprise detecting a change in the level or activity of the metabolic indicator within a microwell. In one embodiment, the level of the metabolic indicator within the sample or microwell prior to separating the sample into the microwell is known or presumed to be zero, such that the detection of appreciable amounts of the metabolic indicator at a later time point after separating the sample into the microwell is indicative of the presence of metabolically active cells in the sample.

In one embodiment, the level of a metabolic indicator is monitored over time. For example, in one embodiment detecting a change in the level or activity of the metabolic indicator comprises:
determining a first level or activity of the metabolic indicator at a first time point;
determining a second level or activity of the metabolic indicator at a second time point;
comparing the first level or activity at the first time point with the second level or activity at the second time point, wherein an increase or decrease in the level or activity of the metabolic indicator indicates the presence of one or more metabolically active cells in the sample.

In one embodiment, a plurality of microwells are monitored over time in order to detect a change or lack thereof in the level or activity of the metabolic indicator across a plurality of microwells. In one embodiment, the method comprises:
determining a first level or activity of the metabolic indicator at a first time point for a plurality of microwells;
determining a second level or activity of the metabolic indicator at a second time point for a plurality of microwells;
comparing the first level or activity at the first time point for the plurality of nanowells with the second level or activity at the second time point for the plurality of microwells.

An increase or decrease in the level or activity of the metabolic indicator in at least one microwell between the first time point and second time point indicates the presence of one or more metabolically active cells in the sample.

In one embodiment, the change in the level or activity of the metabolic indicator arises in the microwell over time as the cell(s) interact with and change the environment within the microwell. The methods and devices described herein allow for the relatively quick detection of cells by detecting changes within the microcell environment over time. The length of time require to alter the microcell environment will depend on the metabolic and/or growth rate of the cell(s) as well as the sensitivity of the detection method for detecting the metabolic indicator. For example, Mycobacterium typically grow more slowly compared to certain species of E. coli and may require monitoring for a longer time in order to detect the presence or absence of metabolically active cells. In one embodiment, the second time point is less than 72 hours, less than 48 hours, less than 24 hours, less than 12 hours, or less than 6 hours from the first time point. In one embodiment, the second time point is less than 4 hours, less than 2 hours, less than 1 hour, less than 30 minutes or less than 15 minutes from the first time point.

Different statistical techniques can be used to determine whether a change has occurred that is indicative of the presence of metabolically active cell within the sample. In one embodiment, the intensity of the signal of the metabolic indicator is integrated or combined over all the microwells containing sample. This will provide a number that is proportional to the concentration of bacteria in the sample. Another approach is to simply count the number of microwells that have a positive signal, irrespective of the intensity. In some embodiments, this provides a simple approach and does not require a sensitive detector to measure signal intensity (such as fluorescence) but is effective in determine the concentration at low sample concentration.

In some embodiments, the methods described herein include incubating the microwells under conditions suitable for growth of the one or more cells between the first time point and the second time point. For example, in some embodiments the sample contains bacteria or is suspected of containing bacteria and the microwells are kept a temperature of about 37 degrees. In other embodiments, the sample contains a fungus or is suspected of containing a fungus and the microwells are kept at a temperature of about 30 degrees.

In one embodiment, the microwells have a volume suitable for containing one or more cells. In one embodiment, the microwells each have a volume between 1 picoliter and 1 microliter. Optionally, the one or more microwells each have a volume between 0.1 nanoliters and 1000 nanoliters. In a preferred embodiment, the one or more microwells each have a volume between 1.0 nanoliters and 100 nanoliters or between 1.0 nanoliters and 1 microliter. In one embodiment, the sample is separated into microwells that have the same volume or have different volumes. In some embodiment, a plurality of microwells are used with different volumes. For example, in one embodiment the sample is separated into one or more microwells of 1.0 nanoliters, one or more microwells of 5 nanoliters, one or more microwells of 10 nanoliters etc. In one embodiment, a plurality of microwells with a gradation of volumes are used in the methods and devices described herein.

As set out in the Examples, in some exemplary embodiments, microwells (100×100) ranging from 100 μm to 1 mm in diameter and a height of 20 μm to 100 μm automatic self-assembly of large sample droplet into individual wells using pneumatic and surface tension assisted methods was observed.

In some embodiments, the one or more microwells are on a microfluidic device. In one embodiment, the microfluidic device comprises a plate with a top surface and the one or more microwells extend below the top surface. Other embodiments may include different arrangements of microwells across one or more surfaces of a microfluidic device. In one embodiment, the surface characteristics and/or materials of the microfluidic device and/or microwells are chosen or altered such as to encourage the separation of a fluid sample into the microwells. For example, in one embodiment, the surface of the microwell is hydrophilic while the area separating adjacent microwells is hydrophobic.

In one embodiment, the microfluidic device comprises a plate with a top surface and one or more microwells extending below the top surface and the top surface is hydrophobic and the surface of the one or more microwells extending below the top surface is hydrophilic.

The microfluidic device described herein may be constructed using techniques known in the art of microdevices such as lithography. For example, in one embodiment the microchannels may be made of polydimethyl siloxane (PDMS), polystyrene, polycarbonate, silicon, glass, polyester, rubber, polyethylene, epoxy, or polyurethane or a combination thereof. In one embodiment, the microfluidic device is made of a material that is amenable to surface modification, such as to include hydrophilic or hydrophobic areas.

In one embodiment, the microwells have a rectangular or square cross-section. In other embodiments, the microwells have an elliptical or circular cross-section. Other microwell shapes suitable for containing one or more cells may also be used with the methods and devices described herein. In one embodiment, the microwells have a diameter between 10 μm and 10,000 μm, between 50 μm and 5,000 μm or between 100 μm and 1000 μm.

In one embodiment, the methods and device described herein use an oxygen sensitive metabolic indicator. Preferably, the microfluidic device comprises a material that prevents diffusion of oxygen into, or out of, the microwells. In one embodiment, all or part of the microfluidic device comprises a material with a gas permeability for oxygen of less than 2.5×10⁻⁵ cm²/s, less than 2.5×10⁻⁶ cm²/s, less than 2.5×10⁻⁷ cm²/s or less than 2.5×10⁻⁸ cm²/s.

In some embodiments, optical methods are used to detect a change in the level or activity of a metabolic indicator within the microwell. Accordingly, in one embodiment, all or part of the microfluidic device forming the microwell is optically transparent. In one embodiment, the metabolic indicator is a fluorophore and all of part of the microwell is optically transparent in the emission wavelength of the fluorophore. In some embodiments, the microwells include a waveguide or light path that allows for a spectra to be obtained of the sample within the microwell.

In one embodiment, the methods and devices described herein use a plurality of microwells in order to separate a sample into a plurality of volumes. In one embodiment, the plurality of microwells comprises at least 10, 50, 100, 500, 1000, 2500 or 10,000 microwells. In one embodiment, the plurality of nanowells comprises between 50 and 10 million nanowells. In some embodiments, methods and devices described herein use a plurality of microwells in an array. In some embodiments, detecting a change in the level or activity of the metabolic indicator comprises detecting a change in the level of the metabolic indicator in a plurality of microwells in the array. In one embodiment, the array comprises at least 5, 10, 20, 50, 100, 200, 500 or 1000 microwells. In one embodiment, the array comprises between 10 and 10⁸ microwells per cm². In one embodiment, the array comprises at least 5, 10, 20, 50, 100, 200, 500, 1000 or 10,000 microwells per cm².

In one embodiment, the microwells contain a liquid such as a growth media. The growth media and/or sample may be put into the microwells using different microfluidic techniques known in the art, including those described in the Examples. Optionally, the growth media is put into the microwells before the sample, with the sample, or after the sample has been separated into the one or more microwells. In one embodiment, the growth media is mixed with the sample prior to separating the sample into one or more microwells.

In one embodiment, the growth media is selective for the growth of a particular cell type. For example, in one embodiment the growth media is selective for a particular pathogen, such that the detection of metabolically active cells indicates the presence of the particular pathogen in the sample. In one embodiment, the growth media is selective for mycobacteria and the presence of one or more metabolically active cells in the sample indicates that the sample contains mycobacteria.

In some embodiments, the microwells and/or growth media may contain one or more antibiotics or test compounds. In one embodiment, the microwells and/or growth media comprises an antibiotic and the presence of one or more metabolically active cells in the sample indicates that the sample contains cells that are resistant to the antibiotic.

In one embodiment, the methods and devices described herein may be used for screening test compounds to identify those compounds that inhibit the metabolic activity or growth of a cell type. For example, in one embodiment the methods and devices described herein may be used to screen compounds for antibiotic activity. In one embodiment, the sample contains a bacterial cell culture, such as a cell culture of a specific pathogenic bacteria at a known concentration or titer. In one embodiment, test compounds that inhibit the metabolic activity or growth of cells in the microwells are identified as having antibiotic activity.

In some embodiments, a sensor is embedded or contained within the microwell for detecting a change in the level or activity of the metabolic indicator. For example, in one embodiment the sensor is an electrochemical sensor, a thermal sensor or an optical sensor.

In one embodiment, the sample, a test compound, an antibiotic, a metabolic indicator, and/or a growth medium is introduced into one or more microwells. In some embodiments, the sample, test compound, antibiotic, metabolic indicator, and/or a growth medium is introduced using screen printing, ink jet printing or other patterning methods. In some embodiments, certain microwells may receive a greater or smaller amount of a test compound or antibiotic etc. in order to determine the efficacy of the test compound or antibiotic.

### Example 1

### Fabrication and Testing of a Microfluidic Device for Detecting Metabolically Active Cells

Conventional methods for the detection of bacterial viability and drug resistance are either expensive, time consuming, or not definitive, and thus do not provide all the information sought by the medical professionals. Here, a method and associated device for the rapid and accurate detection of bacteria in a sample through miniaturization and parallelization is described. This method is demonstrated with wells of several shapes (square, circle), diameters (100-1000 μm) and depths (≦100 μm). A laboratory strain of E. coli was used as a model pathogen. The integration of the fluorescent oxygen sensor, ruthenium tris(2,2′-diprydl)dichloride hexahydrate (RTDP) as a metabolic indicator, allows for the monitoring of the dissolved oxygen concentration as a measure of bacterial metabolism. Detection time of the bacteria within the microwells can be as fast as a few of hours (4-5 hrs), with concentrations that vary between 10² to 10⁸ cells/mL. More rapid detection times are expected depending on the metabolic activity of the cells, the incubation conditions, and the size of the microwell. By adding a test compound and/or drug such as an antibiotic to the sample or the growth medium and measuring growth by detecting a change in the fluorescence intensity of the metabolic indicator, the method may be used to investigate the effects of one or more test compounds on the metabolism or growth of cells such as bacterial cells and drug resistance.

### Fabrication

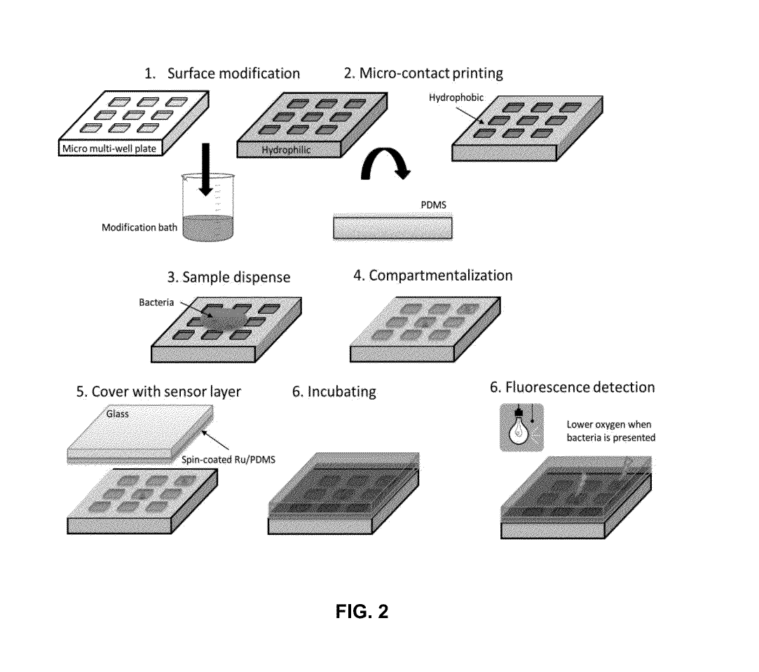

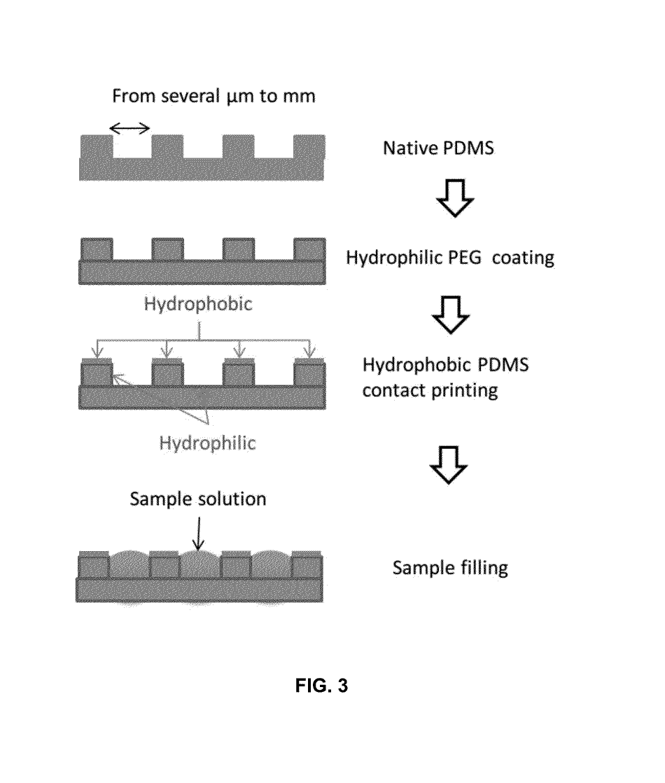

The nano multi-well plates were fabricated through soft-lithography process as shown in FIG. 3. After molding, the surface on the plate is hydrophobic that repels sample solution, therefore a selective surface modification was performed that creates a hydrophilic surface inside each nano well while the rest surface is hydrophobic. The entire nano multi-well plate was immersed in a PEG solution overnight and then rinsed with IPA and DI water. After being dried at ambient temperature, the upper surface (surface outside nano wells) was covered with uncured PDMS by performing micro-contact printing. After the PDMS is cured, the upper surface became hydrophobic while nano well stayed as hydrophilic as shown in FIG. 2. A sample solution of 100 uL which contains targeting bacteria was dispersed on top of the device and subsequently a screen printing-like technique was used to distribute the sample solution into nano wells. In this Example, three different dyes that include tris(4,7-diphenyl-1,10-phenanthroline) ruthenium (II) dichloride (Ru²⁺), methylene blue (MB), and ruthenium tris(2,2′-dipyridyl)dichloride hexahydrate (RTDP) were used to investigate the bacteria metabolism.

### Materials

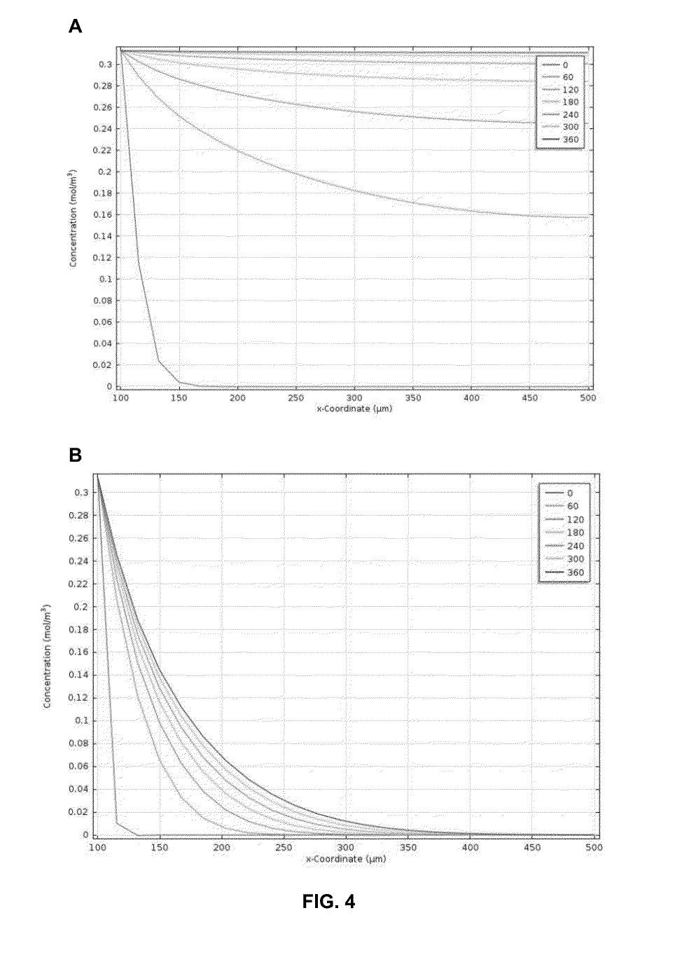

Polydimethylsiloxane (PDMS) and polyurethane (PU) were chosen to fabricate the nano multi-well plates in this Example. PDMS is the most common material used in soft-lithography process for microfluidic devices. Its fabrication process and surface properties are well known and it is also optically transparent for fluorescent imaging. It also has a relatively high gas permeability (2.5×10⁻⁵ cm²/s for oxygen) which allows gases like oxygen to diffuse through easily and could causes interference from adjacent nano wells or ambient. Unlike PDMS, PU is used as moisture and gas barrier and has a much lower gas permeability (2×10⁻⁷ cm²/s for oxygen), therefore could eliminate those possible interference PDMS has. The fabrication of PU-based nano multi-well plate is identical to on reported previously [16] (hereby incorporated by reference in its entirety). A numerical model which has a constant source of concentration (0.31 mol/m³) at the boundary x=100 um when t=0 shows the difference of oxygen diffusion in both PDMS and PU materials. After 360 sec, oxygen has completely diffused into a depth of 500 um in PDMS while little diffusion in PU can be seen in FIG. 4.

### Results

### Compartmentalization

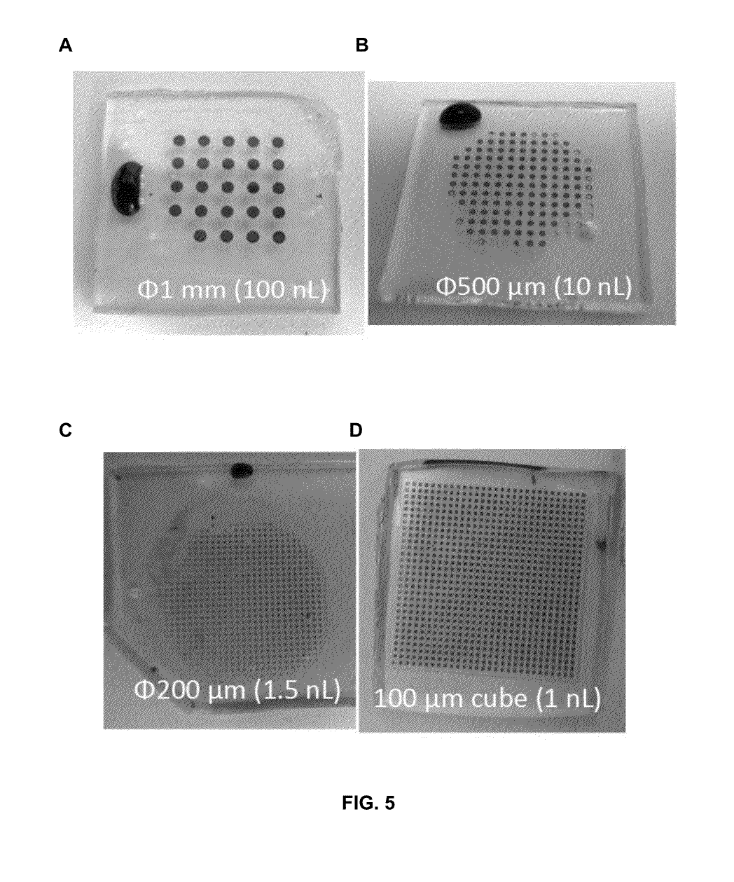

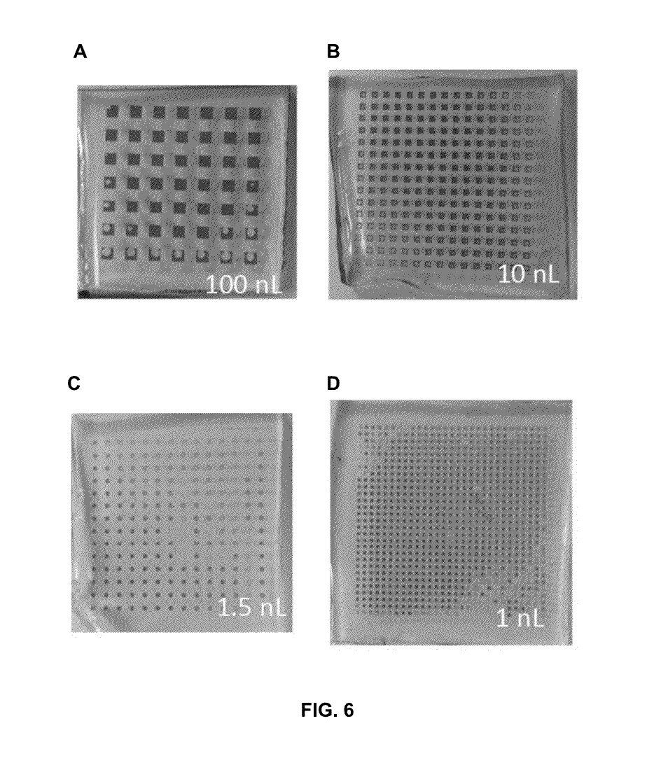

Each bacterium requires certain amount of oxygen for its metabolism. The smaller sample volume within the nano well, the faster change in oxygen concentration can be observed. Therefore, nano multi-well plates with a volume of 100 nL, down to 1 nL, were fabricated in this study. Subsequently, aqueous and protein-rich solutions were used to demonstrate the capability of the nano multi-well plate as shown in FIGS. 5 and 6.

### Viability Study

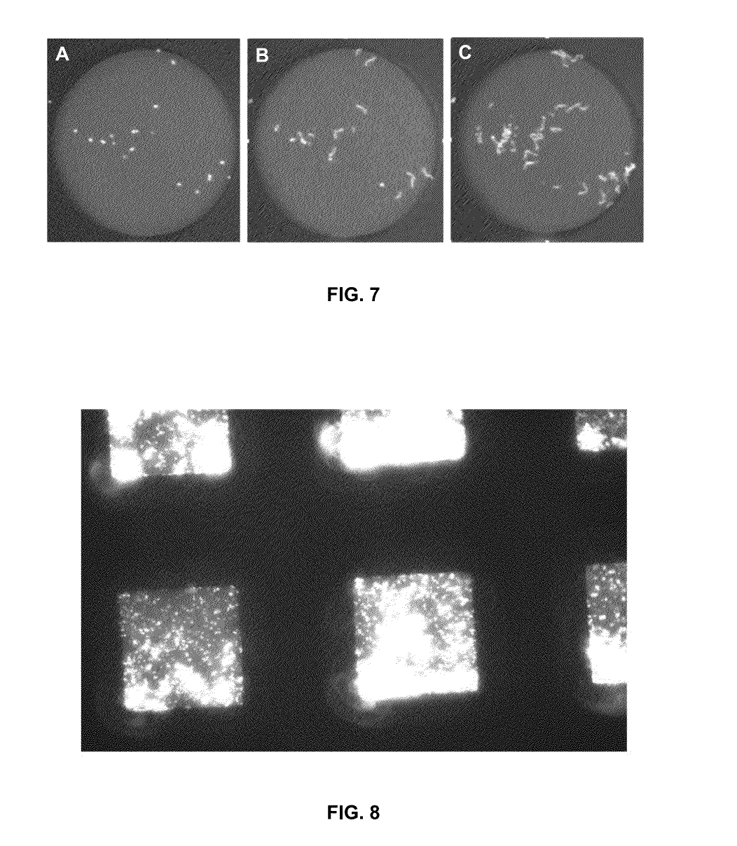

A long-duration incubation of GFP e. coli was performed to ensure the bacterial viability in the micro environment. Bacteria were encapsulated in the well made of PDMS and the growth media was LB solution mixed with 0.3 mg/mL RDTP. Initially there were several isolated bacteria present in the well as shown in FIG. 7(a). FIGS. 7(b) and (c) show the bacteria growth after 2.5 and 5 hr respectively. As time progresses, bacterial division is observed to occur in the wells. Since there is no disturbance in this particular micro environment, the daughter cells were located right next to the mother cells and therefore form in lines. After a 1-day incubation, the overgrown GFP E. coli became a visible mass in the well as shown in FIG. 8. This demonstrates that bacteria remain viabile in the LB/fluorophore solution while the wells were made of PDMS.

### Fluorescent Measurements Using a Plate Reader

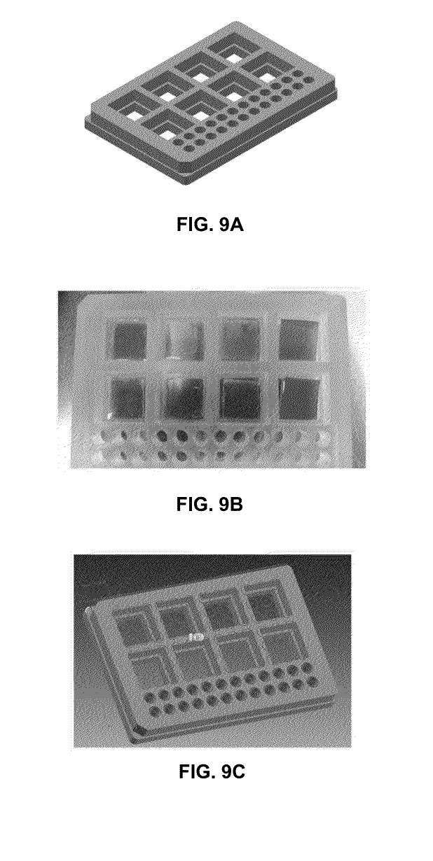

Microplate readers, also known as plate readers, are the standard instruments designed to detect biological events of samples in microtiter plates. In this Example, the feasibility of using nano multi-well plates with microplate readers was investigated. A modified microtiter plate shown in FIG. 9(a) was made using 3D printers in order to fit nano multi-well plates into the microplate readers. The 8 rectangular-shaped holes were designed for nano multi-well plates while the resting 24 circular-shaped holes were identical to 64 well plate as comparison as shown in FIG. 9(b). FIG. 9(c) indicates the relative location between original 96 well plates and the modified microtiter plate. Each nano multi-well plate has one reading from the spot at the center which covers more than 10 microwells depending on the well size.

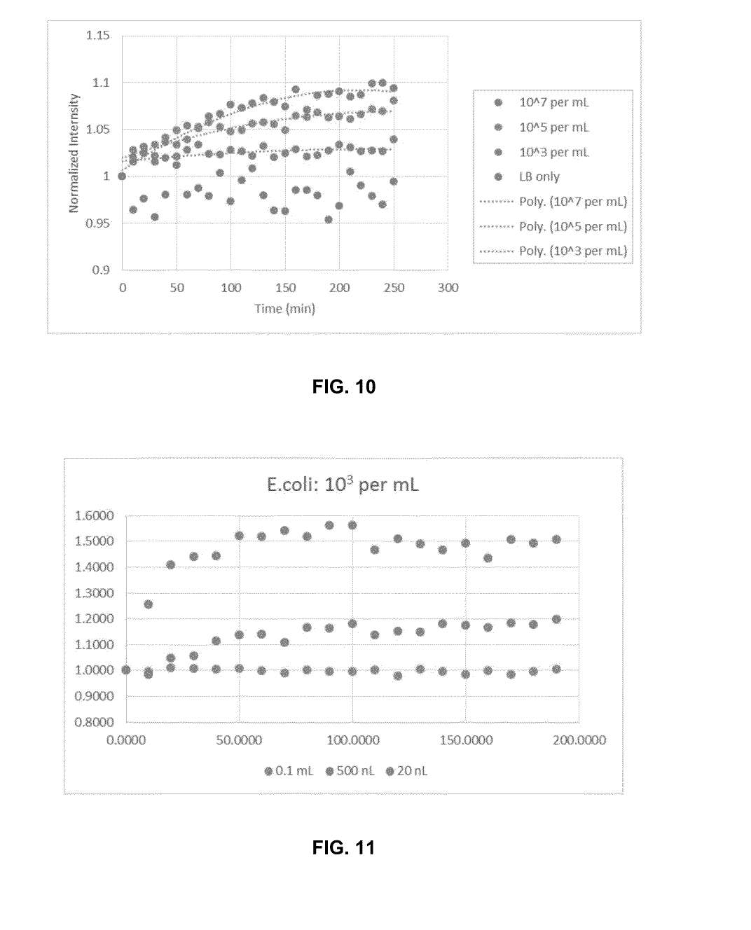

First, a traditional microtiter plate with a sample size of 0.1 mL was used to investigate the effect of bacteria concentration on the response of fluorescence. The dynamic fluorescent intensity from sample solutions with bacteria concentrations ranging from 0 (LB only), 10³, 10⁵ to 10⁷ per mL is shown in FIG. 10. As expected, higher concentration of bacteria resulted in faster fluorescence quenching. Next, the same sample solution with a bacteria concentration of 10³ per mL was dispensed in the traditional microtiter plate (sample size: 0.1 mL) and nano multi-well plate (sample size: 20 nL and 500 nL). As shown in FIG. 11, the fluorescent response from wells with a volume of 20 nL wells was faster than those of 500 nL and 0.1 mL. It is worth mentioning that bacteria may not be present in all micro wells for sample solutions with low bacteria concentration. As mentioned previously, the intensity reading of nano multi-well plate using plate readers may be an average from several micro wells as the spot size is much larger than the size of nano wells. Therefore, the actual fluorescent intensity from a single well where bacteria is present will be higher than this averaged value. Fluorescent microscopy is then required to investigate the dynamic fluorescent response from single well.

### Fluorescent Measurement Using Fluorescent Microscopy

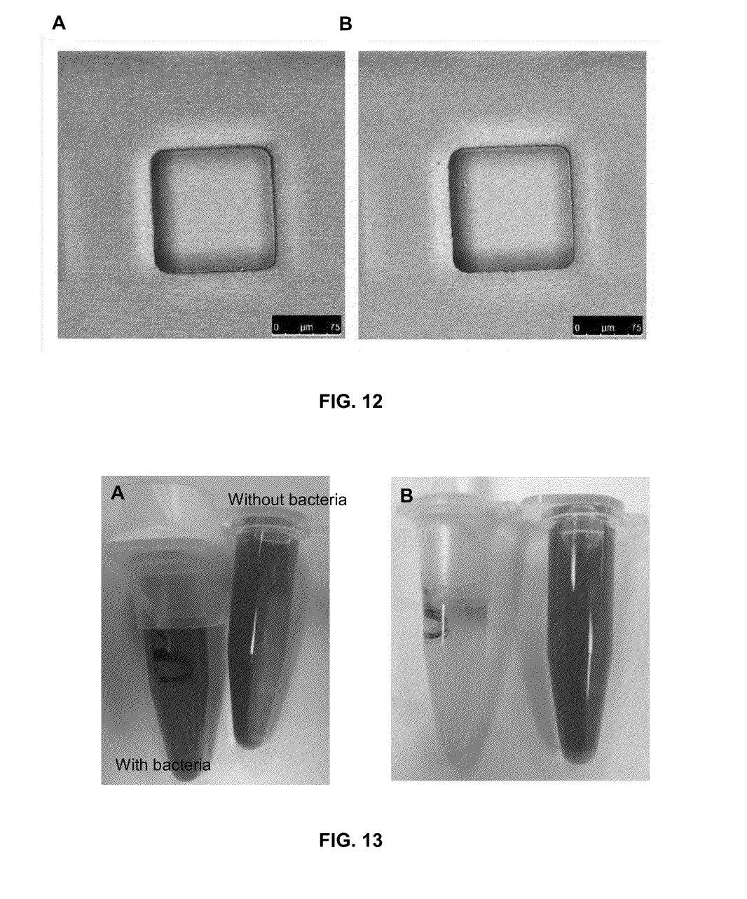

Confocal microscopy (Leica SP5) was used to investigate the fluorescent response within a single well. FIG. 12 shows the fluorescent images by overlapping the fluorescence from the oxygen-sensitive ruthenium complex, the fluorescence from GFP E. coli, and the bright field image shown in red, green and grey respectively.

### Optical Transmission/Absorption Measurement Using MB

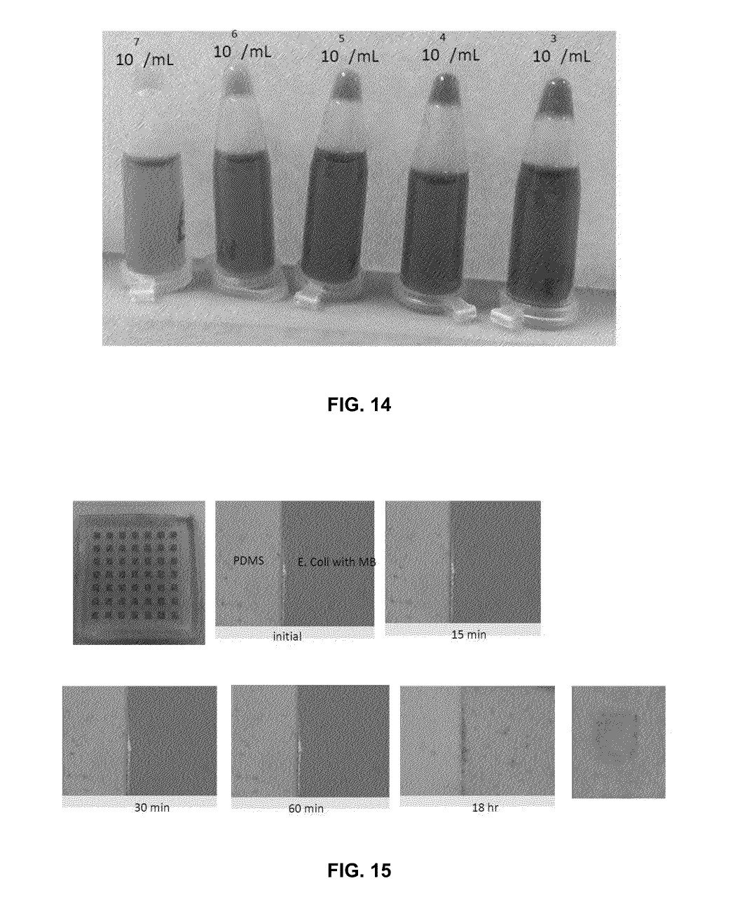

The methylene blue reduction test is mainly used in the dairy industry, and has not been extensively studied with sample solutions other than milk. A preliminary and macro-scale experiment using 1 mL vials was performed to verify the ability of MB to measure E. coli metabolism in LB media. The addition of glucose is required for MB to detect the glycolysis when bacteria are present. Glucose was dissolved in LB media to a concentration of 1 mg/mL while the concentration of bacteria is 10⁸ per mL. In FIG. 13(a), the vial on the left was filled with the solution with the presence of bacteria, and the one on the right was filled with the same solution without bacteria. FIG. 13(b) shows the variation in color after 12 hr incubation and suggests MB was converted from color form into colorless form with the presence of bacteria while the other vial remained as blue. A further study (FIG. 14) also showed the speed of its discoloration is proportional to the amount of bacteria present contained in the solution.

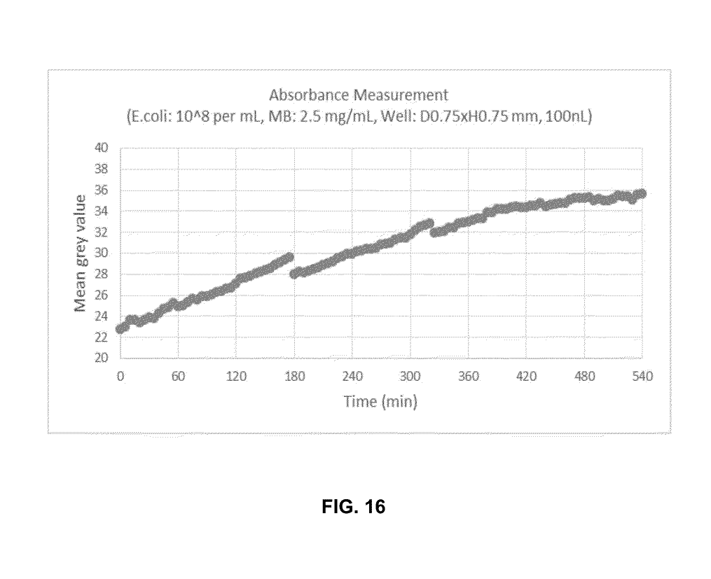

After the of MB for bacteria detection was validated in the macro scale, the same solution with a bacteria concentration of 10⁸ per mL was dispensed on nano multi-well plate with a well size of 100 nL. FIG. 15 shows the discoloration course of MB from t=0 to t=18 hr. The complete discoloration in 100 nL wells happened within 18 hr while it took 24 hr in 1 mL vials. The captured images were then processed using ImageJ to obtain the mean grey scale and the result is shown in FIG. 16.

### Example 2

### Characterization and Testing of Microwells for the Detection of Metabolically Active Cells

### Fabrication

Microfluidic devices were prepared using similar techniques as described in Example 1 and shown in FIG. 2. A silicon wafer was prepared photolithographically with an array of two geometric shapes—either circular or square features. The mask layout was designed in autoCAD (Autodesk Inc., San Francisco, USA) and printed using ultra high-resolution laser photoplotting on transparency sheet. SU-8-100 (80 um thick) negative photoresist (MicroChem Corp., MA, USA) was used to lithographically pattern a master mold of our device. Polydimethylsiloxane (PDMS) pre-polymer mixture (Sylgard 184 kit, Dow Corning Corp., MI, USA; 10:1 ratio of the base and cross-linker) was then cast on the master mold, and cured by placing the device on a hot plate (85 C). The PDMS replica was then peeled off the master mold and cut into pieces containing different sized arrays of microwells. The result is a polymer with an array of wells molded into each of their surfaces (step 1 in FIG. 2). The surface of the microarray is made hydrophilic by incubating it in 10% w/w n-Wet 410 (Enroute Interfaces Inc.) surface modification solution diluted in toluene (step 2 in FIG. 2). Next, PDMS pre-polymer (10:1) was microcontact printed such that only the top surface of the microarray is imprinted and made hydrophobic (step 3 in FIG. 2).

### Experimental Setup and Procedures

In a typical experiment, the sample was mixed with a solution containing a growth medium (Luria-Bertani (LB) medium) and an oxygen sensitive fluorophore (ruthenium tris(2,2′-diprydl)dichloride hexahydrate, (RTDP)—0.1 mg/mL) and dispensed on to the microarray. A simple swiping process dispenses the sample into the hydrophilic microwells while the hydrophobic top surface removes the sample from the top cleanly (step 4 and 5 in FIG. 2). Next, the microarray was capped using a glass slide (made hydrophobic using a surfactant) and imaged under a fluorescent microscope to measure the intensity of the fluorophore.

### Bacterial Culturing

Two types of Escherichia coli strains were used throughout this study. In experimentation requiring imaging the bacteria, a derivative of wild-type E. coli strain K12 (MG1655) expressing green fluorescent protein (GFP) was used as the strain of choice. In other instances, the OP50 E coli strain was used. The OP50 strain is sensitive to ampicillin and its growth rate is reduced. Both strains were cultured in LB media till an OD₆₀₀ of 1.0 corresponding to a concentration of 8.0×10⁸ per mL was obtained. Lower bacterial concentrations of the sample were obtained by serial dilution.

### Results

### Compartmentalization

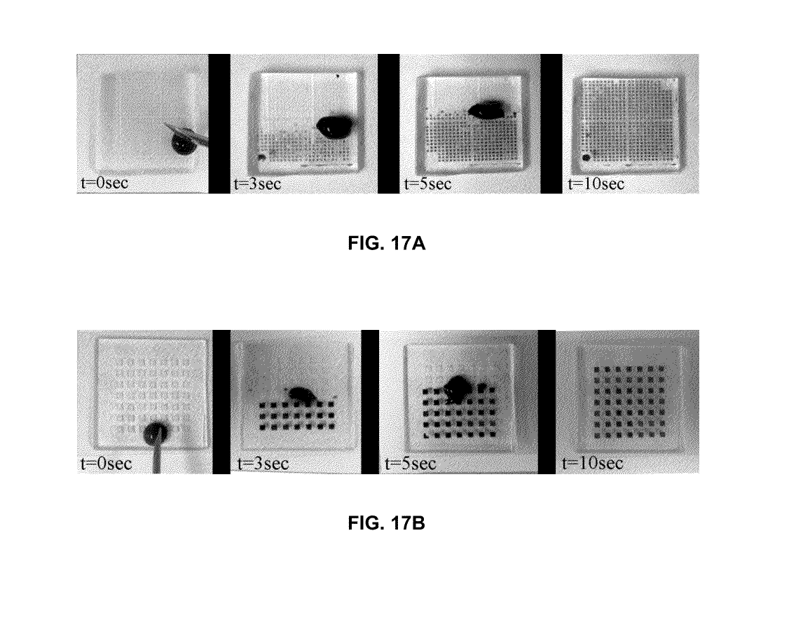

Surface wetting behavior was investigated as a means to segment the sample into thousands of smaller volumes precisely. Various devices with well sizes between 100-1000 μm, were filled using a method in which a droplet of bacterial solution is placed on the surface of the device and using a coverslip to swipe, the liquid is pushed into and compartmentalized within the wells (FIG. 17A, 17B). The difference in wettability of the two surfaces allows for the confinement and easy filling of bacteria containing solution during the filling step.

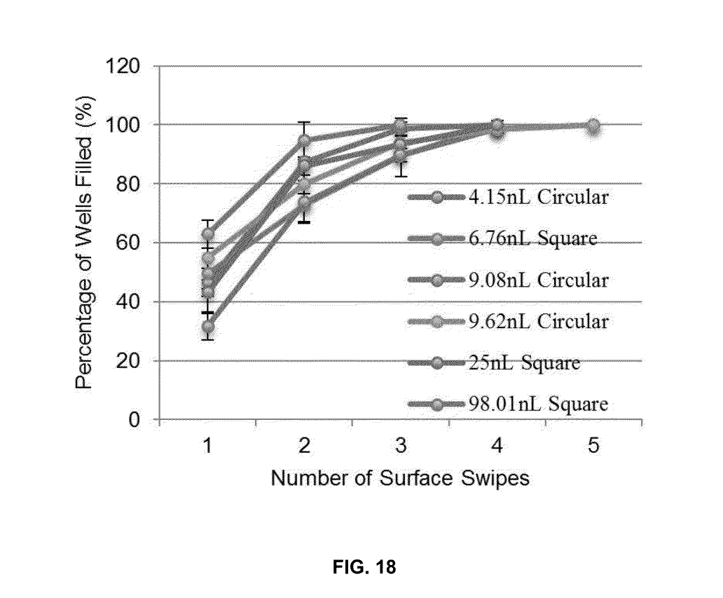

The surface wetting based filling process was found to be effective as shown in FIG. 18. The wells irrespective of their dimension and shape were filled within 4 swipes (back and forth motions). The ability of the sample solution to compartmentalize within the microwell array was studied using liquids of different viscosities ranging from water to high viscosity shear thinning liquids (59.96% water/40% glycerol/0.04% xanthan gum) [21], artificial sputum [22] and protein rich solutions such as milk (mixed with E. coli) were used to study the ability of the liquid to compartmentalize within the wells. It was found that the wells filled in a similar fashion, uniformly, regardless of the viscosity of the solution.

### Measurement of Bacterial Metabolism

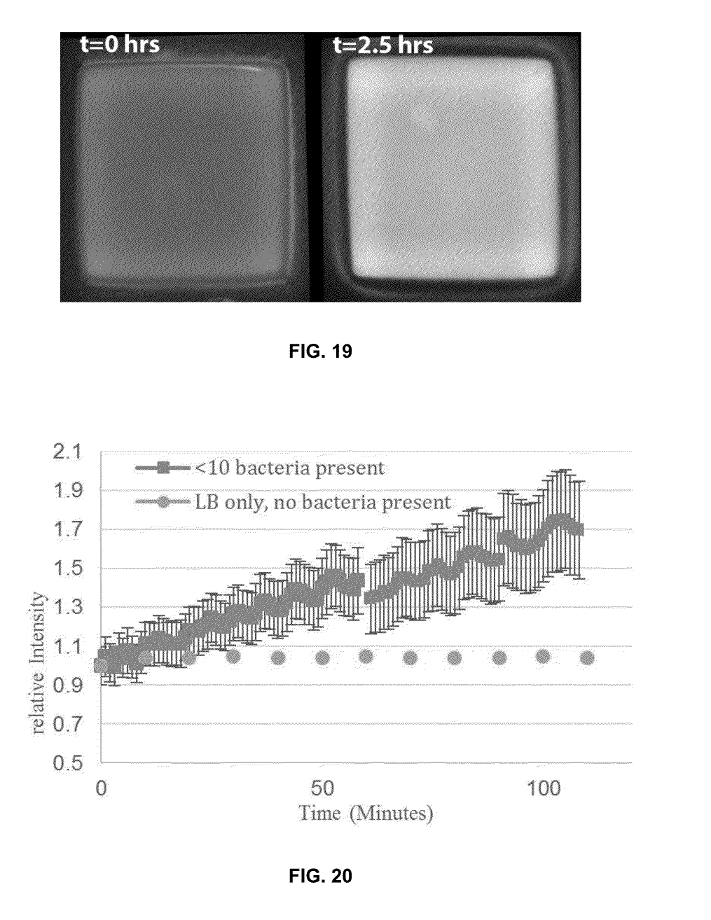

In order to quantitatively measure the metabolism of bacteria and relate it to its growth and viability, the fluorescent metabolic indicator RTDP was used [23]. FIG. 19 shows the change in fluorescent intensity of a single well containing 10⁷ per mL bacteria over 2.5 hours. The fluorescent intensity was observed to increase significantly over time demonstrating the depletion of oxygen due to the metabolism of the bacteria.

In order to study the behavior of aerobic bacterial metabolism within each well, an experiment was conducted in which microwell arrays containing less than 10 bacteria were studied in comparison to arrays of microwells that contained no bacteria. The fluorescent intensity of the microwells that contained bacteria increased rapidly, indicating active metabolism, while those microwells that did not contain the bacteria, did not show an increase in fluorescence (FIG. 20).

Micro well Shape and Size

In order to study the effect of shape and size of the microwells and the response of this sensor, PDMS microwells of various geometries (square or circular) and volumes were fabricated. It was found that the shape of the microwell did not have any significant effect on the rate of fluorescence increase and consequently the speed of detection of bacterial viability.

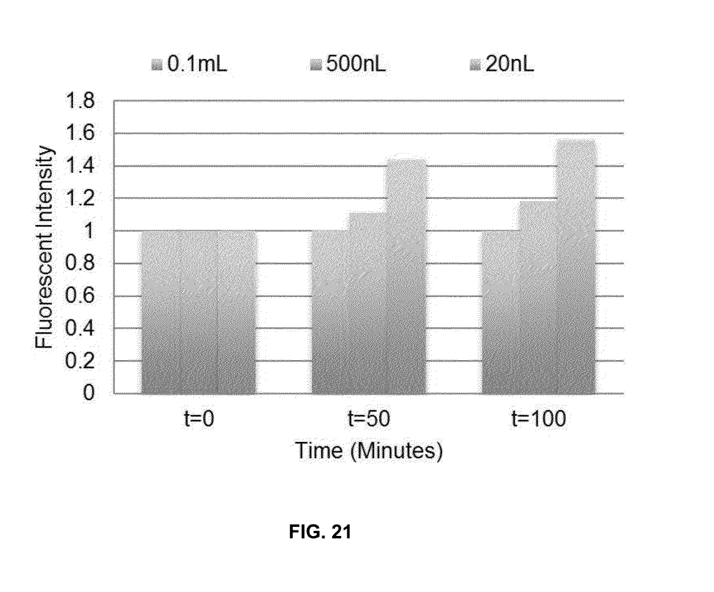

In contrast, the size of the microwells did have a significant effect on the speed of detection. Experiments were conducted where bacterial solutions with concentration of 10⁴ cells/mL were filled into microwell arrays that had individual volumes of 0.1 mL, 500 nL and 20 nL. The measurement of fluorescent intensity measured after 50 and 100 mins (FIG. 21) show that the change in fluorescent intensity increases as the size of the well decreases. This reflects faster detection of viability by segmenting the sample into smaller volumes. It should be noted that microwell volumes below 1 nL were found to be difficult to fill using the segmentation process outlined earlier, whereas microwell volumes that exceeded 100 nL were too large to detect small bacterial metabolism changes within the well. While well sizes between 1 nl and 100 nl appear to be suitable for the detection of E. coli bacteria using the exemplary method and device described herein, other sizes of microwells are expected to be suitable for use with the methods and device provided herein that use different sample solutions or different microfluidic techniques for separating the sample into microwells.

### Drug Resistance

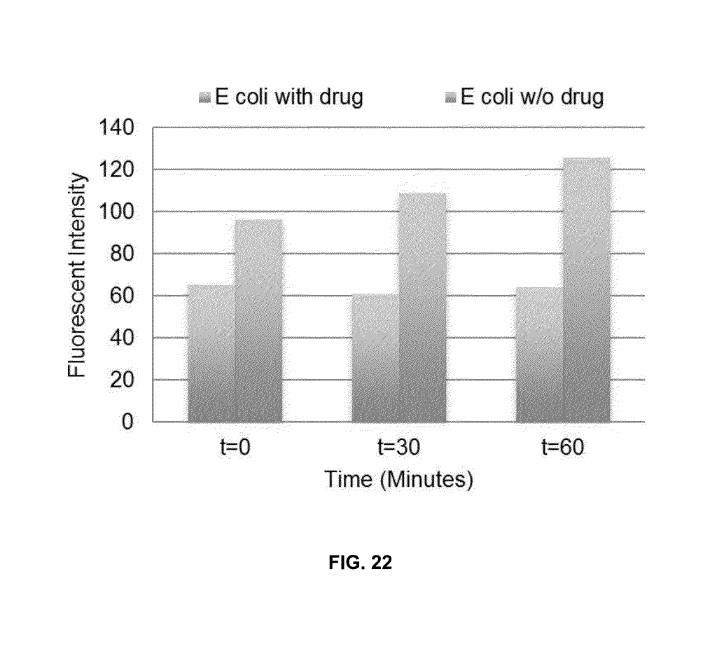

The influence of drugs on the growth of bacteria was investigated using metabolism as a surrogate marker. In this experiment, E. coli strain OP50 (10⁶ cells/mL) was dispersed into the microwells along with the fluorophore RTDP and ampicillin (0.1 mg/mL). Ampicillin is an antibiotic that act as an irreversible inhibitor of the enzyme transpeptidase, which is needed by the OP50 E. coli bacteria to make cell walls, and therefore inhibits growth. After the bacteria were supplied with the drug AMP, the oxygen uptake of the bacteria was analyzed and studied as a direct measure of bacterial viability. As shown in FIG. 22, the fluorescent intensity of the antibiotic free sample increased significantly compared to that with the antibiotic. These results suggest that the growth of the bacteria was inhibited due to the presence of the drug as was expected. The method and device described herein is therefore also expected to be useful for screening compounds for their effect on the metabolism and/or viability of culture cells or bacteria, or for identifying cells or bacteria within a sample that resistant to certain drugs or antibiotics.

The miniaturization and parallelization of microwells as described herein serves as a powerful tool for quickly detecting the metabolic activity of cells. These methods allow for the determination of the growth and drug resistance of bacteria, while keeping the accuracy of the culture test (known as the gold standard). These methods and devices described herein have many applications in different industries and in many different areas such as medicine, food and health safety. The methods and devices described herein are inexpensive, rapid, accurate, and require only minimal sample preparation.

It will be appreciated that certain features of the invention, which are, for clarity, described in the context of separate embodiments or separate aspects, may also be provided in combination in a single embodiment. Conversely, various features of the invention, which are, for brevity, described in the context of a single embodiment or aspect, may also be provided separately or in any suitable sub-combination.

Although the invention has been described in conjunction with specific embodiments thereof, if is evident that many alternatives, modifications and variations will be apparent to those skilled in the art. Accordingly, it is intended to embrace all such alternatives, modifications and variations that fall within the spirit and broad scope of the appended claims. In addition, citation or identification of any reference in this application shall not be construed as an admission that such reference is available as prior art to the present invention.

All publications, patents and patent applications are herein incorporated by reference in their entirety to the same extent as if each individual publication, patent or patent application was specifically and individually indicated to be incorporated by reference in its entirety.

### REFERENCES

[1] Tuberculosis Fact sheet No 104, World Health Organization, November 2010
[2] S. D. Lawn, A. I. Zumla, Tuberculosis, The Lancet, 378, 57-72, 2011.
[3] WHO report of “A ministerial meeting of high M/XDR-TB burden countries,” Beijing, China, 1-3 Apr. 2009
[4] R. Koch, Die aetiologie der tuberkulose. Berliner Klinische Wochenschrift 19; 221-30, 1882.
[5] P. Ehrlich, Referate aus den Verein fur innere Medicin zu Berlin, Deutsche Med Wochenschr, 8, 269, 1882.
[6] F. Ziehl, Ueber die Fairbung des Tuberkelbacillus, Deutsche Med Wochenschr, 9, 247-9, 1883.
[7] F. Neelsen, Ein Casuistischer Beitrag zur Lehre von der Tuberkulose, Zentrablau fpr die medizinischen Wissenschafen, 21, 497-501, 1883.
[8] F. C. Tenover, Diagnostic deoxyribonucleic acid probes for infectious diseases, Clin. Microbiol. Rev. 1, 82-101, 1988.
[9] A. Brisson-Noel, D. Lecossier, X. Nassif, B. Gicquel, V. Levy-Frebault, A. J. Hance, Rapid diagnosis of tuberculosis by amplification of mycobacterial DNA in clinical samples. Lancet, 334, 1069-1071, 1989.
[10] P. W. Hermans, A. R. Schuitema, D. Van Soolingen, C. P. Verstynen, E. M. Bik, J. E. Thole, A. H. Kolk, J. D. van Embden, Specific detection of Mycobacterium tuberculosis complex strains by polymerase chain reaction, J. Clin. Microbiol., 28, 1204-1213, 1990.
[11] D. de Wit, L. Steyn, S. Shoemaker, M. Sogin, Direct detection of Mycobacterium tuberculosis in clinical specimens by DNA amplification. J. Clin. Microbiol. 28, 2437-2441, 1990.
[12] A. Brisson-Noel, C. Aznar, C. Chureau, S. Nguyen, C. Pierre, M. Bartoli, R. Bonete, G. Pialoux, B. Gicquel, G. Garrigue, Diagnosis of tuberculosis by DNA amplification in clinical practice evaluation, Lancet 338, 364-366, 1991.
[13] G. T. Noordhoek, A. H. Kolk, G. Bjune, D. Catty, J. W. Dale, P. E. Fine, P. Godfrey-Faussett, S. N. Cho, T. Shinnick, S. B. Svenson, Sensitivity and specificity of PCR for detection of Mycobacterium tuberculosis: a blind comparison study among seven laboratories, J. Clin. Microbiol., 32, 277-284, 1994.
[14] D. Helb, M. Jones, E. Story, et al., Rapid detection of mycobacterium tuberculosis and rifampin resistance by use of on-demand, near-patient technology, J. Clin. Microbiol., 48, 229-237, 2010.
[15] C. C. Boehme, P. Nabeta, D. Hillemann, et al., Rapid molecular detection of tuberculosis and rifampin resistance, N. Engl. J. Med., 363, 1005-1015, 2010.
[16] W. Wu, K. Sask, J. Brash, P. R, Selvaganapathy, Polyurethane-Based Microfluidic Devices for Blood Contacting Applications, Lab on Chip, 12, 960-970, 2012.
[17] B. G. Subramani, P. Selvaganapathy, Surface Micromachining of PDMS using sacrificial photoresist, Journal of Micromechanics and Microengineering, 19, 015013 (10pp), 2009.
[18] C. Yu, S. Mutlu, P. Selvaganapathy, C. H. Mastrangelo, F. Svec, J. M. J. Frechet, Flow control valves for analytical microfluidic chips without mechanical parts based on thermally responsive monolithic polymers, Analytical Chemistry, 75, 1958-1961, 2003.
[19] P. Selvaganapathy, E. T. Carlen, C. H. Mastrangelo, Electrothermally actuated inline microfluidic valve, Sensors and Actuators, A: Physical, 104, 275-282, 2003.
[20] S. Upadhyaya, P. Selvaganapathy, Microfluidic drug discovery platform for cell based assays, Lab on Chip, 10, 341-348, 2010.
[21] S. R. Wickramasinghe, et. al., “Mass Transfer in Blood Oxygenators Using Blood Analogue Fluids,” Biotechnol Prog, vol. 18, pp. 867-873, 2002.
[22] I. N. de Kantor, et. al., “Laboratory services in tuberculosis control,” World Health Organization, vol. 1, pp 42-43, 1998.
[23] D. Sud, G. Mehta, K. Mehta, J Linderman, S. Takayama, M. Mycek, “Optical imaging in microfluidic bioreactors enables oxygen monitoring for continuous cell culture,” J. Biomed, vol. 1, no. 5, pp. 050504-050504-3, September 2006.

1. A method for detecting the presence of metabolically active cells in a sample, the method comprising:
separating the sample into one or more microwells,
detecting a change in a level or activity of a metabolic indicator in one or more of the microwells, wherein the change is indicative of the presence of one or more metabolically active cells in the sample.

2. The method of claim 1, wherein the metabolic indicator is a compound that is produced by the metabolically active cells or a compound that is consumed by the metabolically active cells.

3. The method of claim 1, wherein the metabolic indicator is an exogenous compound.

4. (canceled)

5. The method of claim 1, wherein the metabolic indicator is an oxygen sensitive fluorophore.

6. The method of claim 5, wherein the oxygen sensitive fluorophore is selected from the group consisting of a ruthenium-based molecule, a metalloporphyrin-type molecule and a metal halide.

7. The method of claim 6, wherein the ruthenium-based molecule is selected from the group consisting of ruthenium tris(2,2′-diprydl)dichloride hexahydrate (RTDP), 4,7-diphenyl-1,10-phenathroline ruthenium (II) chloride, ruthenium(II)-tris(1,10-phenanthroline) and dichlorotris(1,10-phenanthroline) ruthenium (II) hydrate.

8. The method of claim 1, wherein the metabolic indicator is a substrate for an enzyme produced by the metabolically active cells and a decrease in the level of the substrate is indicative of the presence of metabolically active cells in the sample.

9. The method of claim 1, wherein detecting a change in the level or activity of the metabolic indicator comprises:
determining a first level or activity of the metabolic indicator at a first time point;
determining a second level or activity of the metabolic indicator at a second time point;
comparing the first level or activity at the first time point with the second level or activity at the second time point, wherein an increase or decrease in the level or activity of the metabolic indicator indicates the presence of one or more metabolically active cells in the sample.

10. The method of claim 9, further comprising:
determining a first level or activity of the metabolic indicator at a first time point for a plurality of microwells;
determining a second level or activity of the metabolic indicator at a second time point for a plurality of microwells;
comparing the first level or activity at the first time point for the plurality of nanowells with the second level or activity at the second time point for the plurality of microwells.

11. The method of claim 10, wherein the plurality of microwells is at least 50 nanowells.

12. The method of claim 9, wherein the second time point is less than 24 hours from the first time point.

13. The method of claim 9, wherein the second time point is less than 6 hours from the first time point.

14. The method of claim 1, wherein the one or more microwells have a volume between 1.0 nanoliters and 100 nanoliters.

15. The method of claim 1, wherein the one or more microwells are on a microfluidic device.

16. The method of claim 15, wherein the microfluidic device comprises a plate with a hydrophilic top surface and one or more hydrophilic microwells extending below the top surface.

17. The method of claim 16, wherein separating the sample into the one or more microwells comprises contacting the top surface of the plate with the sample and moving all or part of the sample across the top surface of the plate over the one or more microwells.

18. The method of claim 1, wherein the one or more microwells contain a growth media that is selective for the growth of a particular cell type.

19. The method of claim 16, wherein the growth media is selective for mycobacteria and the presence of one or more metabolically active cells in the sample indicates that the sample contains mycobacteria.

20. The method of claim 1, wherein the one or more microwells contain an antibiotic and the presence of one or more metabolically active cells in the sample indicates that the sample contains cells that are resistant to the antibiotic.

21. The method of claim 1, wherein the level or activity of the metabolic indicator is converted into an electrical, optical, chemical or thermal signal inside the microwells.
